(19) United States
(12) Patent Application Publication
(10) Pub. No.: US 2023/0058429 A1
(43) Pub. Date: Feb. 23, 2023

(54) Wire Holder for Sterile Field

(71) Applicant: Andrea Grissom, Lutz, FL (US)

(72) Inventor: Andrea Grissom, Lutz, FL (US)

(21) Appl. No.: 17/944,292

(22) Filed: Sep. 14, 2022

Related U.S. Application Data
(63) Continuation-in-part of application No. 17/403,965, filed on Aug. 17, 2021.

(51) Int. Cl. A61M 25/02; C09J 7/38

(52) U.S. Cl. CPC A61M 25/02; C09J 7/385; A61M 2025/024

(57) ABSTRACT

A wire manager for managing wires in a sterile field includes a body having a plurality of slots on a front side of the body and an adhesive material disposed on a back side of the body for temporarily adhering the body to a surface of the sterile field. A finger tab is formed on a side surface of the body for facilitating removal of the body from the surface on which the body is installed. The adhesive material provides for adhesion to the surface and removal from the surface by way of lifting the body using the finger tab.

### CROSS-REFERENCE TO RELATED APPLICATION

This application is a continuation in part of U.S. patent application Ser. No. 17/403,965, filed Aug. 17, 2021, the disclosure of which is hereby incorporated by reference.

### FIELD

This invention relates to the field of interventional radiology and cardiac catheterization lab and more particularly to a system for managing wires at a sterile field.

### BACKGROUND

When performing various types of procedures, for example, in an interventional radiology room, there are often many catheters or wires used. Some catheters or wires, for example, are for performing the actual procedure, including catheters, guide wires, glide wires, etc.

What is needed is a system that will manage and organize wires in a sterile environment such as a procedural room.

### SUMMARY

In one embodiment, an apparatus for managing wires in a sterile field is disclosed including a body having a plurality of slots on a front side of the body and an adhesive material disposed on a back side of the body for temporarily adhering the body to a surface of the sterile field. A finger tab is formed on a side surface of the body for facilitating removal of the body from the surface on which the body is installed. The adhesive material provides for adhesion to the surface and removal from the surface by way of lifting the body using the finger tab.

In another embodiment, a method of managing wires in a sterile field includes before a procedure, affixing a body of a wire manager to a surface within the sterile field by way of an adhesive material on a back surface of the body and inserting a wire into a slot of a plurality of slots that are formed in the body. Then, during the procedure, removing the wire from the slot. After the procedure, pulling the wire manager from the surface using a finger tab that is formed in a side surface of the body.

In another embodiment, a wire manager for managing wires in a sterile field is disclosed including a body that is planar and has a width, a depth, and a height. An adhesive is disposed on a back surface of the body for temporarily adhering the body to a surface of the sterile field. There are a plurality of slots on a front surface of the body, each of the plurality of slots is sizes for accepting a loop of wire and there is a finger tab disposed on a side surface of the body for facilitating removal from the surface of the sterile field. The adhesive is strong enough to keep the wire manager from moving when the wire manager is on the surface of the sterile field and the adhesive is weak enough for removal of the wire manager from the surface of the sterile field without damaging the wire manager.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a schematic view of a mess of wires in a sterile field such as a procedural table.

FIG. 2 illustrates an elevational view of the wire holder for a sterile field with a wire.

FIG. 3 illustrates an expanded view of the wire holder for the sterile field with the wire.

FIG. 4 illustrates a top-plan view of the wire holder for a sterile field.

FIG. 5 illustrates a bottom-plan view of the wire holder for a sterile field.

FIG. 6 illustrates a perspective view of the wire holder for a sterile field with wires.

FIG. 7 illustrates a perspective view of an alternate version of the wire holder for a sterile field.

FIG. 8 illustrates a top elevational view of the alternate version of the wire holder for a sterile field.

FIG. 9 illustrates a bottom elevational view of the alternate version of the wire holder for a sterile field.

FIG. 10 illustrates a side elevational view of the alternate version of the wire holder for a sterile field.

FIG. 11 illustrates a second side elevational view of the alternate version of the wire holder for a sterile field.

FIG. 12 illustrates a front plan view of the alternate version of the wire holder for a sterile field.

FIG. 13 illustrates a back plan view of the alternate version of the wire holder for a sterile field.

FIG. 14 illustrates a side cut-away view of the alternate version of the wire holder for a sterile field.

FIG. 15 illustrates a front plan view of the alternate version of the wire holder for a sterile field with wires.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

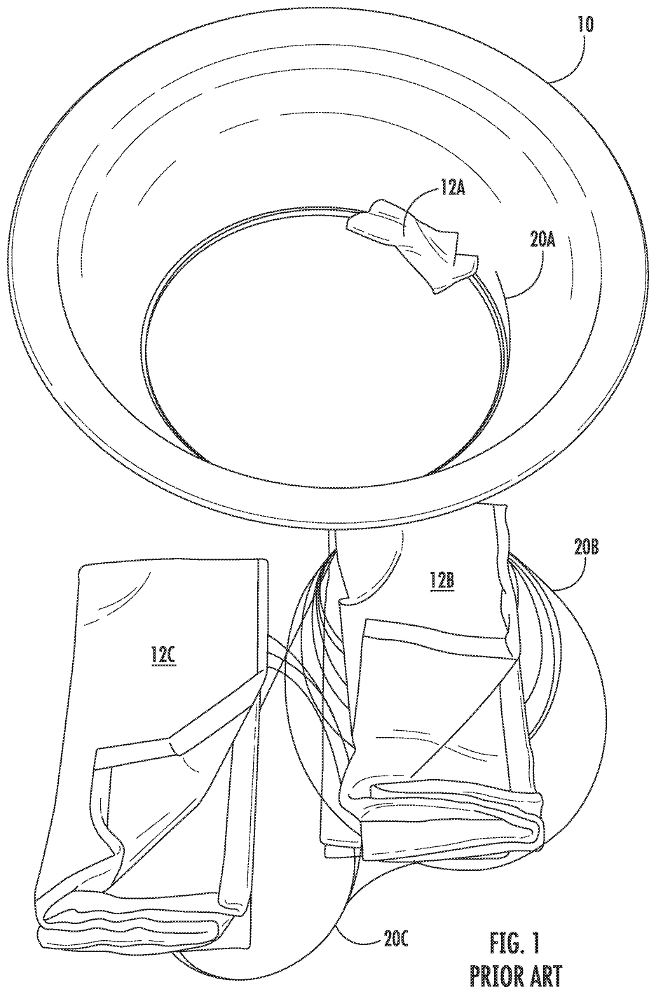

Referring to FIG. 1, a schematic view of a mess of wires of the prior art is shown in a sterile field such as a procedural table. Present day procedural rooms have difficulty managing wires as exemplified in an article titled “Surgery Is An Organized Chaos Of Cords, Tubes And Wires,” Sep. 12, 2011, https://getbetterhealth.com/surgery-is-an-organized-chaos-of-cords-tubes-and-wires/. As shown in FIG. 1, there are three wires 20A/20B/20C. An attempt to keep the wires 20A/20B/20C separated is made using silicone pads 12A/12B/12C and a pan 10, but such silicone pads 12A/12B/12C and pan 10 creates other issues in the sterile field such as they occupy critical space, introduce more objects that might create an infection, require disposal after the procedure or sterilization before and after the procedure, etc. As silicone pads 12A/126/12C are often disposed in sterile disposal receptacles after the procedure, the silicone pads 12A/126/12C incur costs to purchase and costs for disposal, not to mention the effect on the environment.

The above notwithstanding, as visible in FIG. 1, the attempts to organize the wires 20A/206/20C results in chaos in the operating environment a possible tangling which will delay any response to a need for one of the wires 20A/206/20C for detangling.

Note that throughout this description, the term “wires” refers to any elongated, flexible device such as wires, guide wires, catheters, etc.

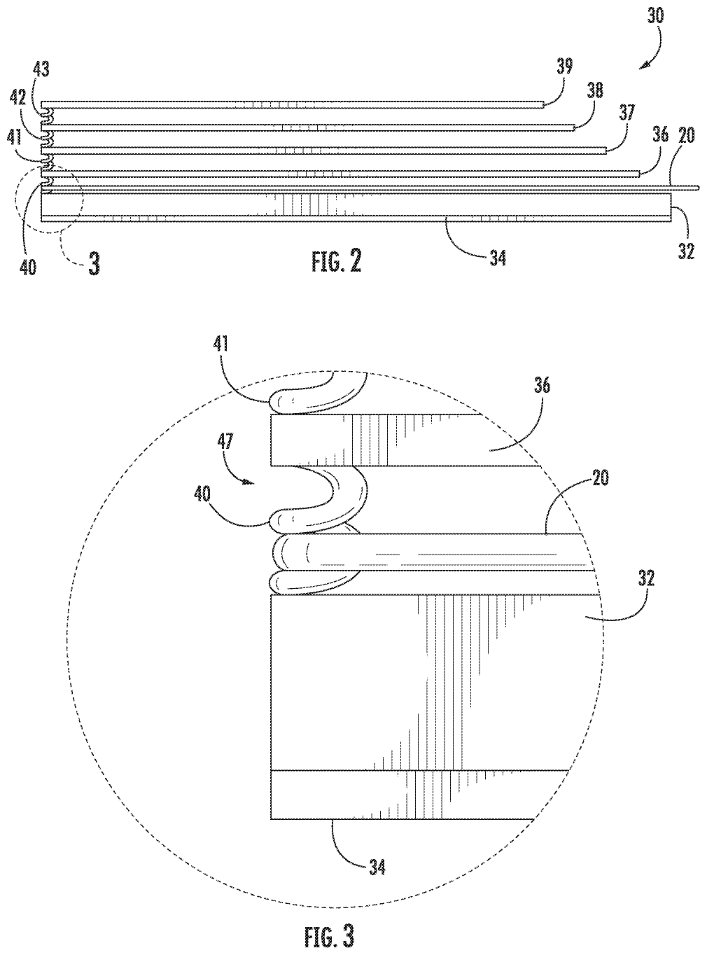

Referring to FIGS. 2 and 3, views of the wire holder 30 for a sterile field with a wire 20 are shown. The wire holder 30 has a base 32 and several separators 36/37/38/39 between which wires 20 are maintained until needed. In some embodiments, the base 32 is planar and made of a material that is suitable for a sterile field (e.g., a procedure room) and is either stiff or resilient (e.g., slightly bendable but returns to an original shape after the force that caused bending abates). One such material is anticipated to be silicone. In some embodiments, the separators 36/37/38/39 are planar to match the base 32, but each of the separators 36/37/38/39 are not necessarily of the same width and depth as the base 32, allowing for ease of indexing during use.

A bottom surface of the base 32 is coated with an adhesive layer 34 (e.g., an adhesive pad, adhesive pads, continuous sheet of adhesive material, adhesive coating). As the adhesive layer 34 is sticky and is used to hold the wire holder 30 to a surface such as a table on which the procedure is performed or a drape on a sterile field, it is preferred that the adhesive layer 34 be made of a repositionable adhesive (sticky material) or pressure-sensitive adhesive such as Pressure Sensitive Acrylate or microsphere adhesives.

The separators 36/37/38/39 are interfaced at one end by wire capture loops 40/41/42/43 that have one or more indentations 47 for capturing the wires 20 (see FIG. 3 in which the wire 20 passes through the indentations 47). Note that although two of the indentations 47 are shown in the figures, any number of the indentations 47 are anticipated, including one indentation 47. The wire capture loops 40/41/42/43 connect the separators 36/37/38/39 to each other at an edge of the separators 36/37/38/39 and one wire capture loop 40 connects the bottom separator 36 to the base 32. Note that in some embodiments, the wire capture loops 40/41/42/43 are made from a material such as silicone and bonded or molded to the separators 36/37/38/39 and base 32 while in other embodiments, the wire capture loops 40/41/42/43 are molded together with the separators 36/37/38/39 and base 32, made from the same material, for example, silicone.

In some embodiments, the base 32 is thicker than the separators 36/37/38/39, for example, ¼ of an inch, while in other embodiments, the base 32 and separators 36/37/38/39 are of the same thickness.

In some embodiments, successive separators 36/37/38/39 are shorter than the prior separators 36/37/38/39 as shown in FIG. 2, allowing easier access to each layer of separators 36/37/38/39, while in other embodiments all separators 36/37/38/39 are of the same dimension.

It is preferred that the separators 36/37/38/39 be made of a thickness that provides for flexibility and resilience so that each separator 36/37/38/39 will bend upwards (in a direction away from the base 32) for insertion/removal of the wires 20 under force (e.g., force of a user's fingers) then, upon abatement of such force, the separator 36/37/38/39 restores to a flat configuration by way of resiliency, assuming that no object such as the wire 20 impedes restoration to the planar configuration.

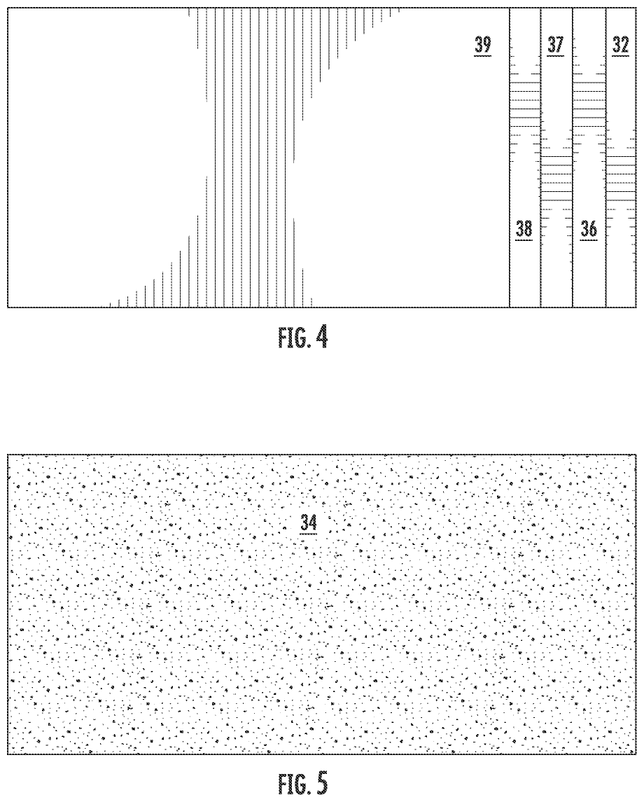

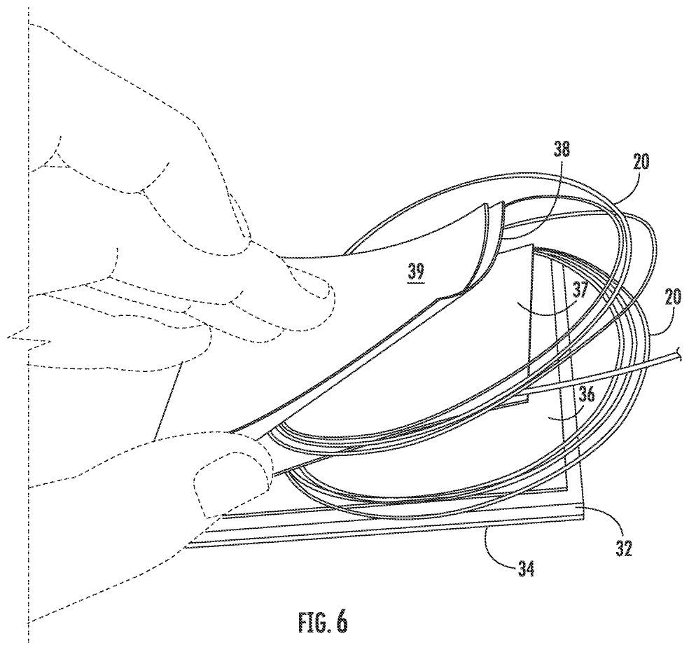

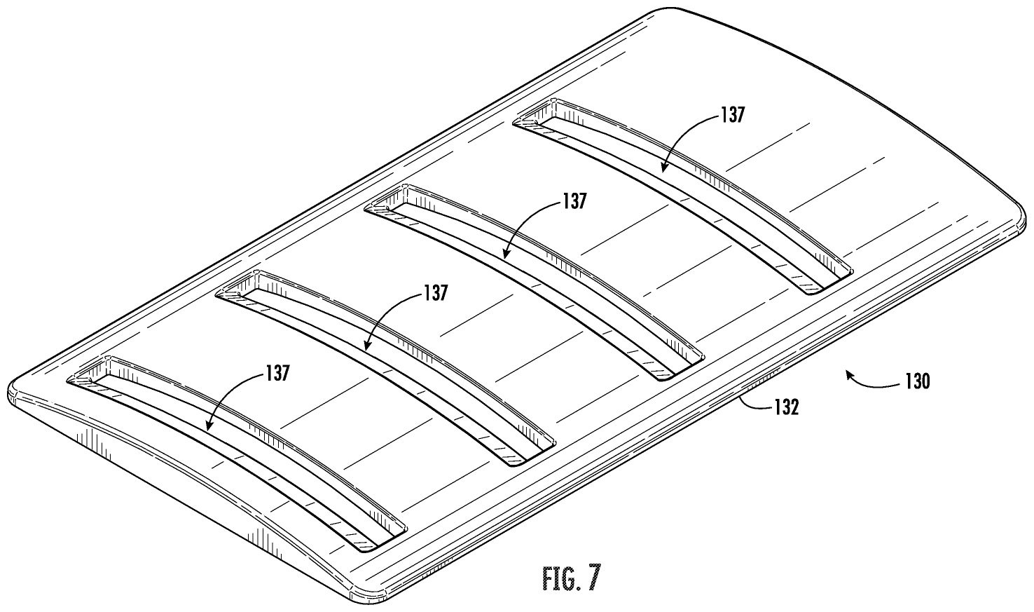

Referring to FIG. 4, a top-plan view of the wire holder 30 for a sterile field is shown. As shown in this embodiment, the separators 36/37/38/39 are planar to match the base 32, and each of the separators 36/37/38/39 are of the same width as the base 32 but vary in depth, allowing for ease of indexing during use as shown in FIG. 6. In this, the top separator 39 (furthest from the base 32) has the shortest depth and the bottom separator 36 has the longest depth, almost as long as the depth of the base 32.

Referring to FIG. 5, a bottom-plan view of the wire holder 30 for a sterile field is shown. In this bottom view, the entire bottom surface of the base 32 is covered with an adhesive layer 34 (e.g., a continuous sheet of adhesive material or an adhesive coating) per one embodiment of the present invention. Note that in other embodiments, an adhesive pad, several adhesive pads, or a section or sections of adhesive coating are on a bottom surface of the base 32. As discussed, the adhesive layer 34 is used to hold the wire holder 30 to a surface such as a table on which a procedure is performed or a drape on a sterile field. Therefore, in one embodiment, the adhesive layer 34 is made of a repositionable adhesive or pressure-sensitive adhesive such as Pressure Sensitive Acrylate or microsphere adhesives. In other embodiments, the adhesive layer 34 is made of any self-sticking material such as double-sided tape. It is fully anticipated that during shipping and storage, a removable cover be placed over the adhesive layer 34 to prevent accumulation of debris and maintain stickiness.

Referring to FIG. 6, a perspective view of the wire holder 30 for a sterile field with wires 20 is shown. In this view, it is shown how several wires 20 are placed between the separators 36/37/38/39. Note howe well organized are the wires 20. In this embodiment, successive separators 36/37/38/39 are shorter than the prior separators 36/37/38/39, allowing easier access to each layer of separators 36/37/38/39. Also, in this embodiment, the separators 36/37/38/39 are made of a thickness that provides for flexibility and resilience so that each separator 36/37/38/39 will bend upwards (in a direction away from the base 32) for insertion/removal of the wires 20 under force (e.g., force of a user's fingers) then, upon abatement of such force, the separator 36/37/38/39 restores to a flat configuration by way of resiliency, assuming that no object such as the wire 20 impedes restoration to the planar configuration.

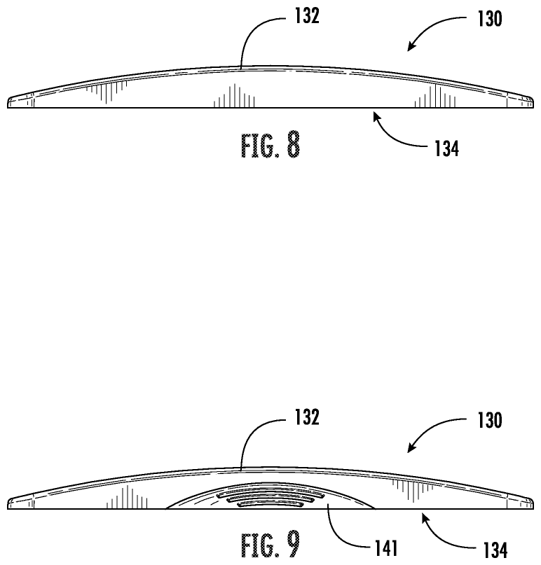

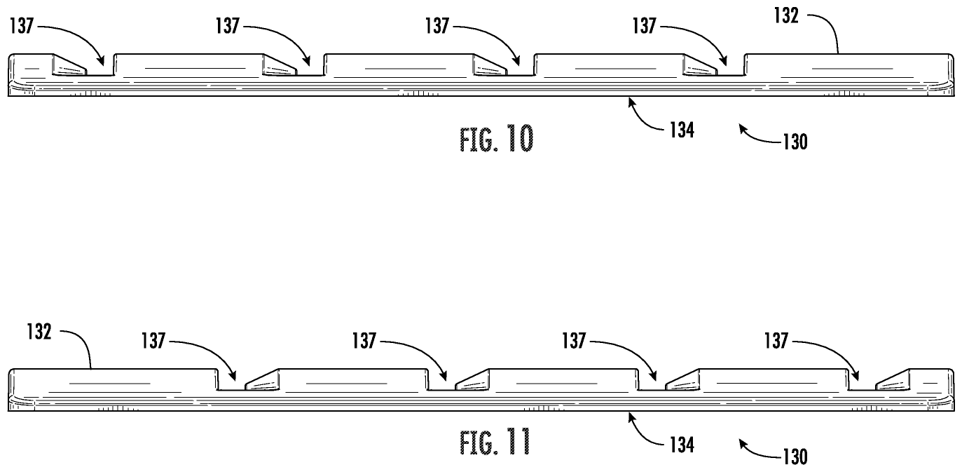

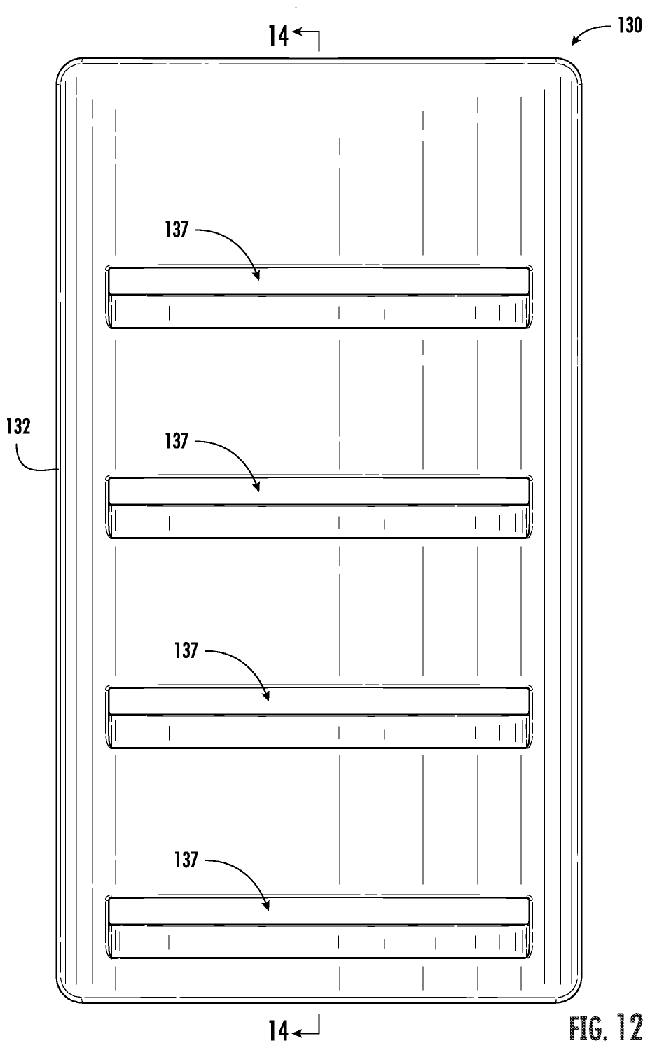

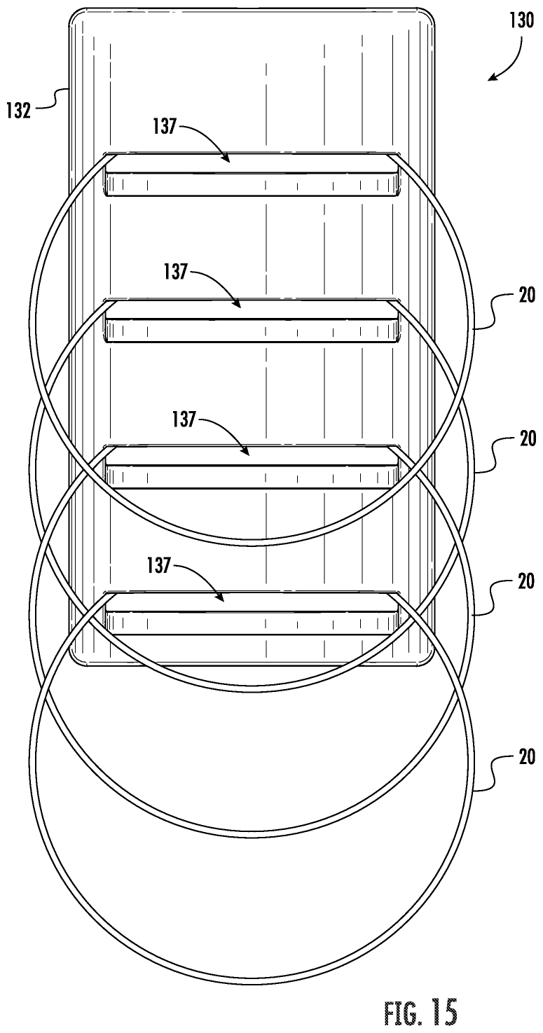

Referring to FIGS. 7-15, an alternate version of the wire holder for a sterile field 130 is shown. The alternate version of the wire holder for a sterile field 130 has a body 132 with a plurality of slots 137 for accepting wires 20 (see FIG. 15) when installed in the sterile field, for example, affixed to an operating table (not shown for clarity and brevity reasons). In some embodiments, one side of each slot 137 is angled so that when wires 20 are placed within the slots 137, the wires 20 lay almost flat with respect to the surface on which the alternate version of the wire holder for a sterile field 130 is installed.

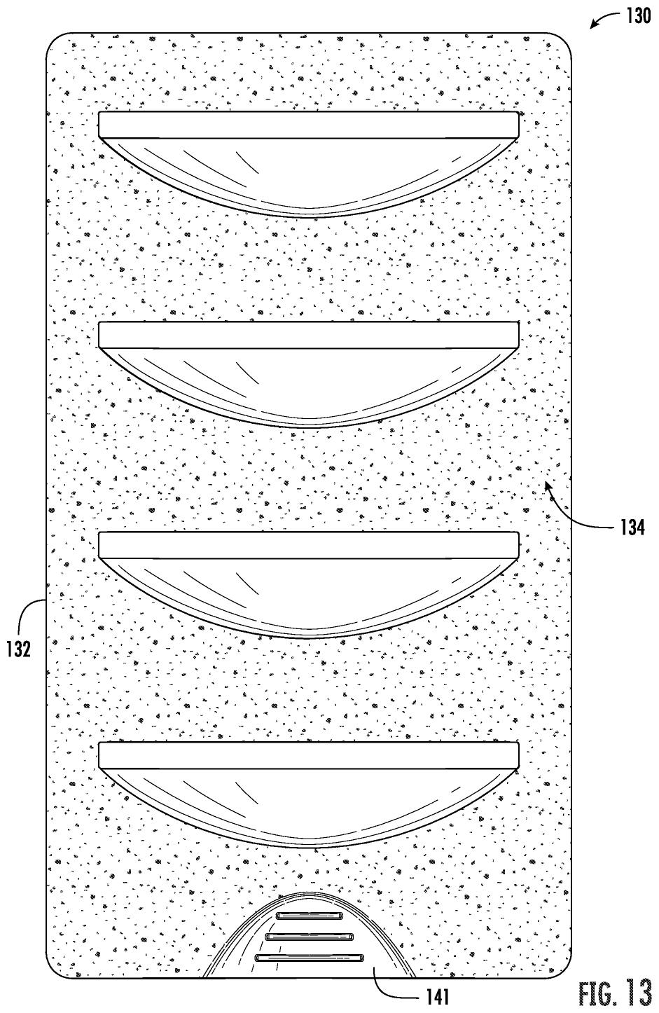

As shown in FIG. 13, the bottom surface of the alternate version of the wire holder for a sterile field 130 is coated (at least partially coated) with an adhesive 134 that holds the alternate version of the wire holder for a sterile field 130, for example, to the operating table. The adhesive 134 is sufficient to hold the alternate version of the wire holder for a sterile field 130, for example, to a stainless-steel surface, yet weak enough to be removed for cleaning and sterilization. On example of such an adhesive is pressure sensitive acrylate.

To facilitate removal, a finger tab 141 allows for a finger or tool to be wedged between the alternate version of the wire holder for a sterile field 130 and the operating table to initiate removal. Note that although an adhesive 134 is shown, it is fully anticipated that the alternate version of the wire holder for a sterile field 130 be held to a surface (e.g., the operating room table) by air pressure by way of the material used to fabricate the alternate version of the wire holder for a sterile field 130 (e.g., a soft, gelatinous plastic or rubber material) or by way of micro suction cups, etc.

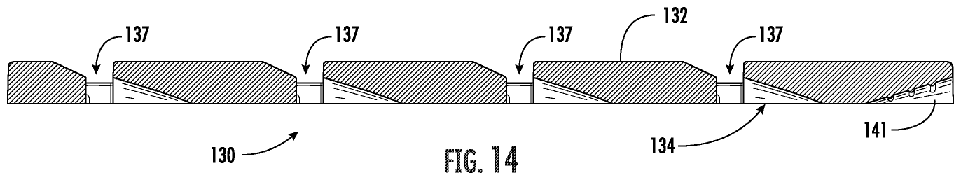

In FIG. 15, it is shown how several wires 20 are placed in the slots 137 for wire management at the sterile field. Note how well the wires 20 are organized. In this embodiment, the wires 20 deform slightly when inserted into the slots 137, thereby holding the wires 20 by frictional force within the slots 137 until removed by, for example, the surgeon or assistants during the procedure. As the inside of the alternate version of the wire holder for a sterile field 130 is at least partially hollow as shown in FIG. 14, the wires 20 extend inwardly into the hollow area of alternate version of the wire holder for a sterile field 130.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. An apparatus for managing wires in a sterile field, the apparatus comprising:
a body having a plurality of slots on a front side of the body;
an adhesive material disposed on a back side of the body for temporarily adhering the body to a surface of the sterile field;
a finger tab formed on a side surface of the body for facilitating removal of the body from the surface on which the body is installed; and
whereas the adhesive material provides for adhesion to the surface and removal from the surface by way of lifting the body using the finger tab.

2. The apparatus for managing wires of claim 1, wherein the body is made of silicone.

3. The apparatus for managing wires of claim 1, wherein the adhesive material is selected from a group consisting of an adhesive pad, a plurality of adhesive pads, a continuous sheet of adhesive, and an adhesive coating.

4. The apparatus for managing wires of claim 1, wherein the adhesive material is one or more suction cups.

5. The apparatus for managing wires of claim 1, wherein the adhesive material is pressure sensitive acrylate.

6. A method of managing wires in a sterile field during a procedure, the method comprising:
before the procedure, affixing a body of a wire manager to a surface within the sterile field by way of an adhesive material on a back surface of the body and inserting a wire into a slot of a plurality of slots that are formed in the body;
during the procedure, removing the wire from the slot; and
after the procedure, pulling the wire manager from the surface using a finger tab that is formed in a side surface of the body.

7. The method of claim 6, wherein the body is made of silicone.

8. The method of claim 6, wherein the adhesive material is selected from a group consisting of an adhesive pad, a plurality of adhesive pads, a continuous sheet of adhesive, and an adhesive coating.

9. The method of claim 6, wherein the adhesive material is one or more suction cups.

10. The method of claim 6, wherein the adhesive material is pressure sensitive acrylate.

11. A wire manager for managing wires in a sterile field, the wire manager comprising:
a body that is planar and has a width, a depth, and a height;
an adhesive disposed on a back surface of the body for temporarily adhering the body to a surface of the sterile field;
a plurality of slots on a front surface of the body, each of the plurality of slots is sizes for accepting a loop of wire;
a finger tab disposed on a side surface of the body for facilitating removal from the surface of the sterile field; and
whereas the adhesive is strong enough to keep the wire manager from moving when the wire manager is on the surface of the sterile field and the adhesive is weak enough for removal of the wire manager from the surface of the sterile field without damaging the wire manager.

12. The wire manager of claim 11, wherein the body is made of silicone.

13. The wire manager of claim 11, wherein the adhesive is selected from a group consisting of an adhesive pad, a plurality of adhesive pads, a continuous sheet of an adhesive material, and an adhesive coating.

14. The wire manager of claim 11, wherein the adhesive is pressure sensitive acrylate.

15. The wire manager of claim 11, wherein the adhesive is at least one suction cup.

16. The wire manager of claim 14, wherein the height of the body is greater than the width of the body.
